(19) United States
(12) Patent Application Publication
(10) Pub. No.: US 2025/0328686 A1
(43) Pub. Date: Oct. 23, 2025

(54) ARTIFICIAL INTELLIGENCE-BASED ADVANCED SECURE INFORMATION SYSTEMS AND METHODS

(71) Applicant: State Farm Mutual Automobile Insurance Company, Bloomington, IL (US)

(72) Inventors: Edward W. Breitweiser, Bloomington, IL (US); Jennifer L. Crawford, Normal, IL (US); Ryan K. Mathews, New Albany, OH (US); Kenny Haggard, Bloomington, IL (US); Ashley Peters, Cottage Grove, MN (US)

(21) Appl. No.: 19/169,269

(22) Filed: Apr. 3, 2025

Related U.S. Application Data
(60) Provisional application No. 63/635,385, filed on Apr. 17, 2024.

(51) Int. Cl. G06F 21/62; G16H 10/60; G16H 50/20

(52) U.S. Cl. CPC G06F 21/6245; G16H 10/60; G16H 50/20

(57) ABSTRACT

A computer system for coordinating advanced secure information is provided. The system is programmed to: a) store a plurality of sets of personal information for a plurality of individuals, wherein each set of personal information of the plurality of personal information is stored with a plurality of privacy settings; b) receive a request for access to a first set of personal information for a first individual, wherein the request for access includes one or more attributes; c) compare the one or more attributes of the request for access to the plurality of privacy settings for the first set of personal information; d) determine one or more items of information from the first set of personal information approved to be provided in response to the request for access; and/or e) generate and transmit a response to the request for access to the first set of personal information.

### CROSS-REFERENCE TO RELATED APPLICATIONS

This application claims priority to U.S. Provisional Patent Application No. 63/635,385, filed Apr. 17, 2024, entitled “SYSTEMS AND METHODS FOR ADVANCED SECURE INFORMATION SYSTEMS,” the entire contents of which is hereby incorporated herein by reference in its entirety.

### FIELD OF THE DISCLOSURE

The present disclosure relates to advanced secure information systems and methods, and more particularly, to a network-based system and method for using artificial intelligence (AI) tools to analyze past secure information interactions to determine optimal communications and delivery system with individual users.

### BACKGROUND

In several jurisdictions, personal information is protected by laws and/or regulations. In some of these jurisdictions, the personal information may be actually owned by the subject rather than the holder of the information. Furthermore, the security of this information is important and may lead to penalties if not properly protected. Accordingly, it would be useful to determine what information is being held about each individual subject, where that information is being stored, and to be able to provide that information to the subject upon request. Conventional techniques may have other efficiencies, encumbrances, ineffectiveness, and/or drawbacks as well.

### BRIEF SUMMARY

The present embodiments may relate to, inter alia, advanced secure information methods, systems and delivery systems with an individual, and more particularly, to a network-based system and method for using artificial intelligence (AI) tools to analyze past secure information interactions to determine optimal communications and delivery system with individual users. The systems and method may be configured to secure personal information and provide portions of that information to requesting parties. The systems and methods described herein may provide for analyzing a plurality of personal information and providing recommendations based upon that analysis to each individual user.

In one aspect, a computer system configured to utilize artificial intelligence tools to protect and provide secure information may be provided. The computer system may include one or more local or remote processors, servers, sensors, memory units, transceivers, mobile devices, wearables, smart watches, smart glasses or contacts, augmented reality glasses, virtual reality headsets, mixed or extended reality headsets, voice bots, chatbots, ChatGPT bots, and/or other electronic or electrical components, which may be in wired or wireless communication with one another. For instance, the computer system may include a computing device that may include at least one processor in communication with at least one memory device. The at least one processor may be configured to: (1) store a plurality of sets of personal information for a plurality of individuals, wherein each set of personal information of the plurality of personal information is stored with a plurality of privacy settings for accessing the corresponding set of personal information; (2) receive, from a requestor device, a request for access to a first set of personal information for a first individual, wherein the request for access includes one or more attributes; (3) compare the one or more attributes of the request for access to the plurality of privacy settings for the first set of personal information; (4) determine one or more items of information from the first set of personal information approved to be provided in response to the request for access; (5) generate a response to the request for access to the first set of personal information including the one or more items of information; and/or (6) transmit, to the requestor device, the response to the request for access to the first set of personal information. The computer system may include additional, less, or alternate functionality, including that discussed elsewhere herein.

In another aspect, a computer-implemented method for protecting and providing secure information may be provided. The computer-implemented method may be performed by a computer device including at least one processor in communication with at least one memory device. The method may include: (1) storing a plurality of sets of personal information for a plurality of individuals, wherein each set of personal information of the plurality of personal information is stored with a plurality of privacy settings for accessing the corresponding set of personal information; (2) receiving, from a requestor device, a request for access to a first set of personal information for a first individual, wherein the request for access includes one or more attributes; (3) comparing the one or more attributes of the request for access to the plurality of privacy settings for the first set of personal information; (4) determining one or more items of information from the first set of personal information approved to be provided in response to the request for access; (5) generating a response to the request for access to the first set of personal information including the one or more items of information; and/or (6) transmitting, to the requestor device, the response to the request for access to the first set of personal information. The computer-implemented method may include additional, less, or alternate actions, including those discussed elsewhere herein.

In another aspect, at least one non-transitory computer-readable media having computer-executable instructions embodied thereon may be provided. When executed by a computing device including at least one processor in communication with at least one memory device, the computer-executable instructions may cause the at least one processor to: (1) store a plurality of sets of personal information for a plurality of individuals, wherein cach set of personal information of the plurality of personal information is stored with a plurality of privacy settings for accessing the corresponding set of personal information; (2) receive, from a requestor device, a request for access to a first set of personal information for a first individual, wherein the request for access includes one or more attributes; (3) compare the one or more attributes of the request for access to the plurality of privacy settings for the first set of personal information; (4) determine one or more items of information from the first set of personal information approved to be provided in response to the request for access; (5) generate a response to the request for access to the first set of personal information including the one or more items of information; and/or (6) transmit, to the requestor device, the response to the request for access to the first set of personal information. The computer-executable instructions may direct additional, less, or alternate functionality, including that discussed elsewhere herein.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed herein. However, it should be understood that the present embodiments are not limited to the precise arrangements and/or instrumentalities shown herein.

FIG. 1 illustrates a block diagram of an exemplary privacy management system for analyzing past privacy interactions to determine effective and/or optimal privacy settings for individual users, in accordance with at least one embodiment.

FIG. 2 illustrates an exemplary computer implemented process for analyzing past privacy interactions to determine effective and/or optimal privacy settings for individual users using the system shown in FIG. 1.

FIG. 3 illustrates an exemplary computer system for performing the processes shown in FIG. 2.

FIG. 4 is a schematic diagram of an exemplary privacy analysis (PA) server shown in FIG. 1, that may be used with the systems shown in FIGS. 1 and 3.

FIG. 5 illustrates an exemplary configuration of a user computer device, in accordance with one embodiment of the present disclosure.

FIG. 6 illustrates an exemplary configuration of a server computer device, in accordance with one embodiment of the present disclosure.

The Figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION OF THE DRAWINGS

The present embodiments may relate to, inter alia, a network-based system and method for coordinating advanced secure information systems, and more particularly, to a network-based system and method for using artificial intelligence (AI) tools to analyze past secure information interactions to determine optimal communications and delivery system with individual users. In one exemplary embodiment, the process may be performed by a privacy analysis (PA) computer device. The PA computer device may be configured to protect and provide secure information in accordance with certain privacy settings set by the individual associated with the secure information wherein the information may be provided to different requestors of the information.

In the exemplary embodiment, the PA computer device may be in communication with one or more user devices, one or more analysis models, one or more internal data sources, one or more external data sources, and/or one or more external requestor systems. As described below in further detail, the PA computer device may include one or more large language models (LLM), such as GPT (Generative Pre-trained Transformers) models, and one or more supplemental models that are configured to curate data from internal and external sources to send to the one or more GPT models. The one or more supplemental models are configured to leverage the one or more GPT models for their wide range of capabilities. In some embodiments, the systems and methods described herein may also use behavioral models and/or economic models in addition to models based upon conversations.

At least one goal of the systems and methods described herein is to determine the best way (e.g., most effective or optimal delivery system for providing only needed private information to requestors) to determine which items of information to provide to requestors. This includes determining the information requested by the requestor, determining the information provided to similar requestors in the past, determining privacy settings of the individual whose information is being requested, and/or determining which information to provide to the requestor. This process may be learned from a plurality of interactions, such as, but not limited to, previous requests for private information and the responses to those requests, and/or privacy settings of other individuals with similar attributes to the corresponding individual. These interactions are used to help train the models that are then used to output recommendations for privacy settings and provided information.

In the exemplary embodiment, the PA computer device may be in communication with one or more databases containing personal and/or private information. For example, the one or more databases may contain PII (personally identifiable information. The one or more databases may also contain personal healthcare information (PHI). The PA computer device is also in communication with one or more large language models (LLM), such as GPT (Generative Pre-trained Transformers) models that allow the PA computer device to make determinations as described herein. This may include the PA computer device identifying optimal privacy settings for the personal information of an individual based on the privacy settings of other similar individuals and the personal information of the individual. In some further embodiments, this may also include the PA computer device analyzing the personal information of the individual to determine one or more recommendations to the individuals. The personal recommendations may include products and/or services that would be suggested for the individual. The personal recommendations may also include healthcare recommendations to make to the individual to potentially improve their health and/or to prevent potential health conditions.

In at least one embodiment, the personal information may include personal healthcare information (PHI) that has been gathered through interactions with the corresponding individual. In some embodiments, the PHI is collected in interactions between the individual and an insurance provider. The PHI may include information collected during exams to apply for different services, such as, but not limited to, life insurance. Furthermore, the PHI may include information collected in claims, such as from an automobile accident. In some further embodiments, the PHI may also include family PHI from other related individuals.

In the exemplary embodiment, the PA computer device may control access to the PHI of the individual. The PA computer device allows the individual to see what information is available about that individual and to control access of others to that information. In the exemplary embodiment, the PA computer device has access to a plurality of privacy settings for the personal information of each individual. The plurality of privacy settings control access to that personal information by those other than the individual. The privacy settings may also control what personal information different users may access. In some embodiments, this may be set by the category of individual. For example, if a job application requests some information, then the individual may authorize the requested information be provided, but no other information may be provided. In another example, if the individual is visiting a new doctor, the individual may give that doctor access to all of the healthcare information, just information related to the doctor's specialty, or only information within a specific period of time.

In some embodiments, the individual sets the plurality of privacy settings. In other embodiments, the plurality of privacy settings are controlled by a slider with different privacy settings being associated with different values on the slider. In some further embodiments, the PA computer device is in communication with one or more LLMs that analyze privacy settings. The one or more LLMs analyze the privacy settings of other individuals and compare those individuals to the individual in question. Then the one or more LLMs output recommended privacy settings for the individual in question. These privacy settings may be presented to the individual and/or automatically applied to the individual's privacy settings. In other embodiments, the one or more LLMs determine the appropriate privacy settings for cach position on the privacy slider.

In some further embodiments, the PA computer device allows access to the personal information of an individual to other computer devices via advanced application programming interfaces (APIs).

In additional embodiments, the PA computer device may be in communication with one or more LLMs that are trained to analyze healthcare data. These LLMs analyze the healthcare data of the individual and determine a current condition of the individual's health. In some embodiments, the LLMs may detect a risk to the individual's health based on the analyzed personal healthcare information. The PA computer device may then transmit a recommendation to the individual to see a doctor about this potential issue. The LLMs may also provide one or more recommendations to improve the health of the individual and the PA computer device provides those recommendations to the individual.

While the systems and methods described herein disclose insurance-based examples, one having skill in the art would understand that these are for example purposes only and that the systems and methods described herein may be used for other implementations in other industries as well.

### Exemplary Intelligent Privacy Handling System

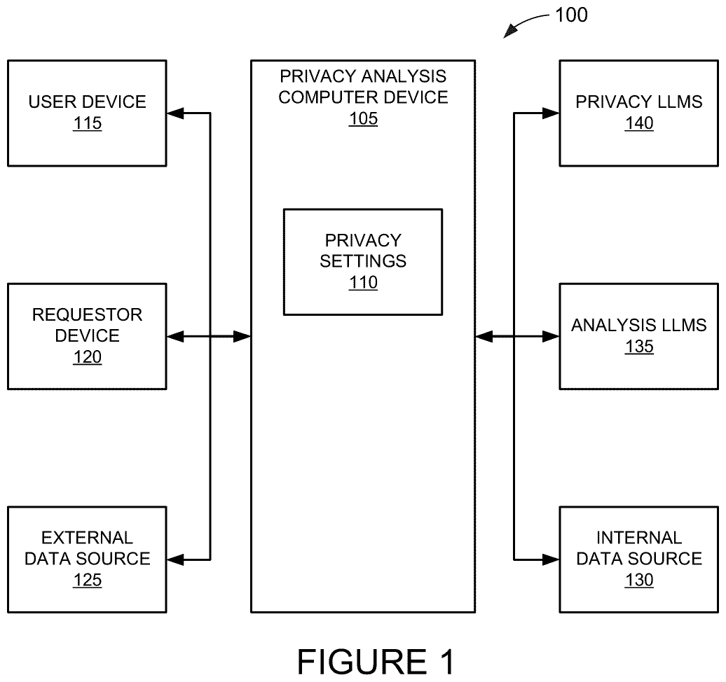

FIG. 1 illustrates a block diagram of an exemplary privacy management system 100 for analyzing past privacy interactions to determine effective and/or optimal privacy settings for individual users, in accordance with at least one embodiment. In the exemplary embodiment, the privacy management system 100 is configured to determine the best way (e.g., most effective or optimal delivery system for providing only needed private information to requestors) to determine which items of information to provide to requestors. This includes determining the information requested by the requestor, determining the information provided to similar requestors in the past, determining privacy settings of the individual whose information is being requested, and/or determining which information to provide to the requestor. This may be learned from a plurality of interactions, such as, but not limited to, previous requests for private information and the responses to those requests, and/or privacy settings of other individuals with similar attributes to the corresponding individual. These interactions are used to help train the models that are then used to output recommendations for privacy settings and provided information.

In the exemplary embodiment, a privacy analysis (PA) computer device 105 is in communication with one or more data sources 125 and 130 containing personal and/or private information of different people. For example, the one or more data sources 125 and 130 may contain PII (personally identifiable information). The one or more data sources 125 and 130 may also contain personal healthcare information (PHI). The PA computer device 105 may also be in communication with one or more large language models (LLM) 135 and 140, such as GPT (Generative Pre-trained Transformers) models that allow the PA computer device 105 to make determinations as described herein. This may include the PA computer device 105 identifying optimal privacy settings 110 for the personal information of an individual based on the privacy settings 110 of other similar individuals and the personal information of the individual. In some further embodiments, this may also include the PA computer device 105 analyzing the personal information of the individual to determine one or more recommendations to the individuals. The personal recommendations may include products and/or services that would be suggested for the individual. The personal recommendations may also include healthcare recommendations to make to the individual to potentially improve their health and/or to prevent potential health conditions.

In at least one embodiment, the personal information may be personal healthcare information (PHI) that has been gathered through interactions with the corresponding individual. In some embodiments, the PHI is collected in interactions between the individual and an insurance provider. The PHI may include information collected during exams to apply for different services, such as, but not limited to, life insurance. Furthermore, the PHI may include information collected in claims, such as from an automobile accident. In some further embodiments, the PHI may also include family PHI from other related individuals.

In the exemplary embodiment, the PA computer device 105 controls access to the PHI of the individual. The PA computer device 105 allows the individual to see what information is available about that individual and to control access of others to that information. In the exemplary embodiment, the PA computer device 105 has access to a plurality of privacy settings 110 for the personal information of each individual. The plurality of privacy settings 110 control access to that personal information by those other than the individual. The privacy settings may also control what personal information different users may access. In some embodiments, this may be set by the category of individual. For example, if a job application requests some information, then the individual may authorize the requested information be provided, but no other information may be provided. In another example, if the individual is visiting a new doctor, the individual may give that doctor access to all of the healthcare information, just information related to the doctor's specialty, or only information within a specific period of time.

In some embodiments, the individual sets the plurality of privacy settings 110. In other embodiments, the plurality of privacy settings 110 are controlled by a slider with different privacy settings being associated with different values on the slider. In some further embodiments, the PA computer device 105 may be in communication with one or more LLMs 140 that analyze privacy settings. The one or more LLMs 140 may analyze the privacy settings 110 of other individuals and compare those individuals to the individual in question. Then the one or more LLMs 140 output recommended privacy settings 110 for the individual in question. These privacy settings 110 may be presented to the individual and/or automatically applied to the individual's privacy settings 110. In other embodiments, the one or more LLMs 140 may determine the appropriate privacy settings for each position on the privacy slider.

In some further embodiments, the PA computer device 105 allows access to the personal information of an individual to other computer devices, such as requestor devices 120 via advanced programming interfaces (APIs).

In additional embodiments, the PA computer device 105 is in communication with one or more LLMs 135 that are trained to analyze healthcare data. These LLMs 135 analyze the healthcare data of the individual and determine a current condition of the individual's health. In some embodiments, the LLMs 135 may detect a risk to the individual's health based on the analyzed personal healthcare information. The PA computer device 105 may then transmit a recommendation to the individual to see a doctor about this potential issue. The LLMs may 135 also provide one or more recommendations to improve the health of the individual and the PA computer device 105 provides those recommendations to the individual customer, determines what the system does not know about the customer, identifies risk factors that are unknown for the customer, and/or identify the most efficient method to gather information from the customer.

In the exemplary embodiment, the privacy management system 100 may include a privacy analysis (PA) computer device 105. The PA computer device 105 may be configured to receive requests for information about a participant, individual, and/or user. The PA computer device 105 may be in communication with one or more trained analysis LLMs 135 and/or privacy LLMs 140. In at least one embodiment, the large language models 135 and 140 may be GPT (Generative Pre-trained Transformers) models.

The PA computer device 105 may also be in communication with one or more user devices 115. The user devices 115 are computer devices being used by an individual to control access to their personal information. In addition, the PA computer device 105 may be in communication with one or more requestor devices 120 associated with different groups requesting one or more items of the individual's personal information. For example, a first requestor device 120 may be associated with an insurance agent setting up an account. A second requestor device 120 may be associated with a doctor looking for the individual's healthcare history and/or family healthcare history. A third requestor device 120 may be associated with a job that the individual is applying for, etc.

In the exemplary embodiment, the PA computer device 105 may receive a request for information about a first individual from the requestor device 120. The request may be for information that would be part of a questionnaire for the first individual to set-up a service, such as life insurance, for that individual. The request may be for information about the first individual to better find out what the first individual's current health condition or health history.

In the exemplary embodiment, the PA computer device 105 may access the privacy settings 110 for the individual to determine which items of personal information that the PA computer device 105 may provide to the requestor device 120. In some embodiment, the personal information is stored in one or more internal data sources 130. In other embodiments, the PA computer device 105 provides access to information in one or more external data sources 125. In still further embodiments, the PA computer device 105 provides information from both external data sources 125 and internal data sources 130.

### Exemplary Computer-Implemented Method

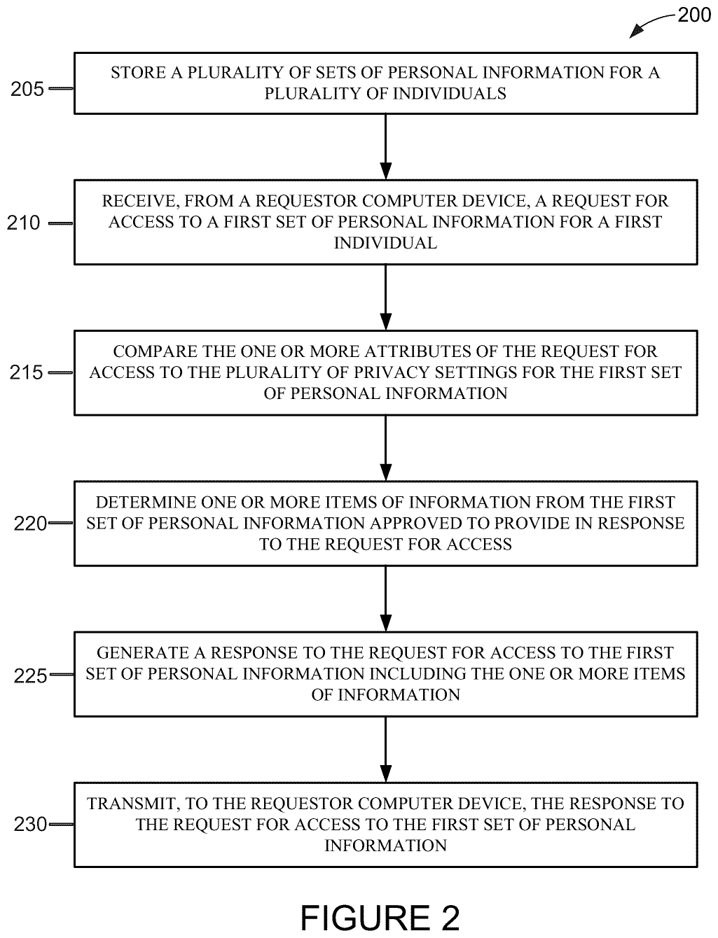

FIG. 2 illustrates an exemplary computer implemented process 200 for analyzing past privacy interactions to determine effective and/or optimal privacy settings for individual users using the system 100 (shown in FIG. 1). In the exemplary embodiment, method 200 may be implemented by the PA computer device 105 (shown in FIG. 1).

In the exemplary embodiment, the PA computer device 105 stores 205 a plurality of sets of personal information for a plurality of individuals. Each set of personal information of the plurality of personal information is stored with a plurality of privacy settings 110 (shown in FIG. 1) for accessing to the corresponding set of personal information.

In the exemplary embodiment, the PA computer device 105 receives 210, from a requestor device 120 (shown in FIG. 1), a request for access to a first set of personal information for a first individual. The request for access includes one or more attributes. These attributes include information about the requestor, such as category, name, etc. In some further embodiments, the request includes a list of one or more items of personal information being requested. The one or more attributes may include a category for a requestor associated with the requestor device 120. The PA computer device 105 may determine one or more items of information from the first set of personal information to provide in response to the request for access based upon the category for the requestor. The PA computer device 105 may determine one or more items of information from the first set of personal information to prevent access to based upon the category for the requestor.

In the exemplary embodiment, the PA computer device 105 may compare 215 the one or more attributes of the request for access to the plurality of privacy settings 110 for the first set of personal information.

In the exemplary embodiment, the PA computer device 105 may determine 220 one or more items of information from the first set of personal information approved to provide in response to the request for access.

In the exemplary embodiment, the PA computer device 105 may generate 225 a response to the request for access to the first set of personal information including the one or more items of information.

In the exemplary embodiment, the PA computer device 105 may transmit 230, to the requestor device 120, the response to the request for access to the first set of personal information.

In some further embodiments, the PA computer device 105 may execute a privacy model 140 (shown in FIG. 1) to determine the one or more items of information from the first set of personal information to approve providing in response to the request for access. The PA computer device 105 may also train the privacy model 140 to determine an amount of access to provide to requestors based upon a plurality of historical requests and responses. The PA computer device 105 may further determine one or more of the plurality of privacy settings 110 based upon execution of the privacy model 140.

In some embodiments, the plurality of personal information may include personal healthcare information (PHI). The PA computer device 105 may analyze a set of personal information to determine a healthcare recommendation for the corresponding individual. The PA computer device 105 may provide the healthcare recommendation to the corresponding individual.

In some further embodiments, the requestor device 120 may execute an application programming interface (API) to transmit the request for access. Additionally or alternatively, the PA computer device 105 may receive the plurality of privacy settings for a set of personal information from the corresponding individual.

In additional embodiments, the PA computer device 105 may analyze a set of personal information to determine a service to provide to the corresponding individual.

### Exemplary Computer System

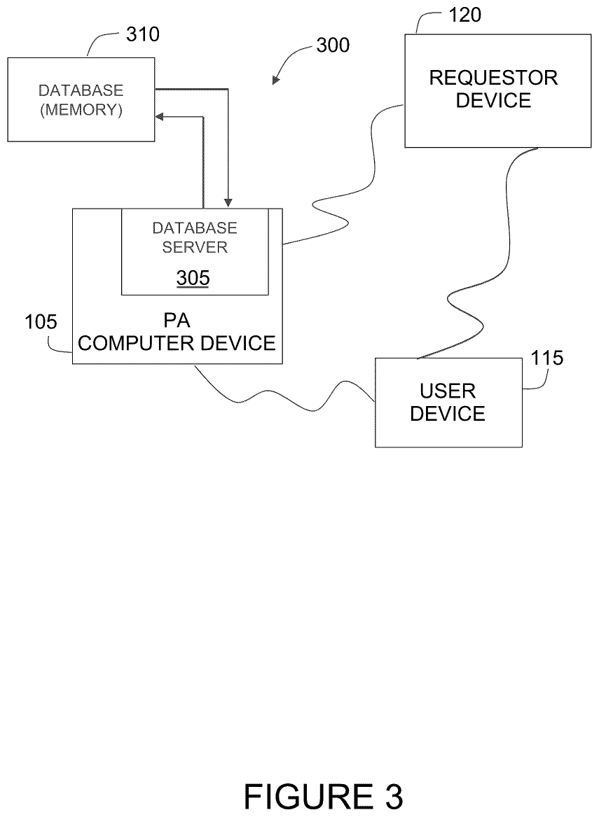

FIG. 3 illustrates an exemplary computer system 300 for performing the process 200 (shown in FIG. 2). In the exemplary embodiment, the system 300 may be used for using artificial intelligence tools to analyze past privacy interactions to determine effective and/or optimal privacy settings for individual users.

As described below in more detail, the privacy analysis (PA) computer device 105 may be programmed for privacy setting analysis. In addition, the PA computer device 105 may be programmed to coordinate the communication and execute of large language models (LLM), such as analysis LLMs 135 and privacy LLMs 140. In some embodiments, the PA computer device 105 may be programmed to: (1) store a plurality of sets of personal information for a plurality of individuals, wherein each set of personal information of the plurality of personal information is stored with a plurality of privacy settings 110 for accessing the corresponding set of personal information; (2) receive, from a requestor device 120, a request for access to a first set of personal information for a first individual, wherein the request for access includes one or more attributes; (3) compare the one or more attributes of the request for access to the plurality of privacy settings 110 for the first set of personal information; (4) determine one or more items of information from the first set of personal information approved to be provided in response to the request for access; (5) generate a response to the request for access to the first set of personal information including the one or more items of information; and/or (6) transmit, to the requestor device 120, the response to the request for access to the first set of personal information.

In the exemplary embodiment, the PA computer device 105 (also known as PA server 105) may be a computer that includes a web browser or a software application, which enables PA computer device 105 to communicate with user devices 115 and requestor devices 120 using the Internet, a local area network (LAN), or a wide area network (WAN). In some embodiments, the PA computer device 105 may be communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem.

PA computer device 105 may be a device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chatbots, voice bots, ChatGPT bots or ChatGPT-based bots, or other web-based connectable equipment or mobile devices.

In the exemplary embodiment, user devices 115 may be computers or computing devices that include a web browser or a software application, which enables user devices 115 to communicate with PA computer device 105 using the Internet, a local area network (LAN), or a wide area network (WAN). In some embodiments, the user devices 115 are communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem. User devices 115 may be a device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chatbots, voice bots, ChatGPT bots or ChatGPT-based bots, or other web-based connectable equipment or mobile devices.

In the exemplary embodiment, requestor devices 120 may be computers or computing devices that include a web browser or a software application, which enables requestor devices 120 to communicate with PA computer device 105 using the Internet, a local area network (LAN), or a wide area network (WAN). In some embodiments, the user devices 115 are communicatively coupled to the Internet through many interfaces including, but not limited to, at least one of a network, such as the Internet, a LAN, a WAN, or an integrated services digital network (ISDN), a dial-up-connection, a digital subscriber line (DSL), a cellular phone connection, a satellite connection, and a cable modem. Requestor devices 120 may be a computing device capable of accessing a network, such as the Internet, including, but not limited to, a desktop computer, a laptop computer, a personal digital assistant (PDA), a cellular phone, a smartphone, a tablet, a phablet, wearable electronics, smart watch, virtual headsets or glasses (e.g., AR (augmented reality), VR (virtual reality), MR (mixed reality), or XR (extended reality) headsets or glasses), chatbots, voice bots, ChatGPT bots or ChatGPT-based bots, or other web-based connectable equipment or mobile devices.

A database server 305 may be communicatively coupled to a database 310 that stores data. In one embodiment, the database 310 may be a database that includes one or more large language models and/or personal information. In some embodiments, the database 310 is stored remotely from the PA computer device 105. In some embodiments, the database 310 is decentralized. In the exemplary embodiment, an individual may access the database 310 via the user devices 145 by logging onto PA computer device 105.

### Exemplary Server Device

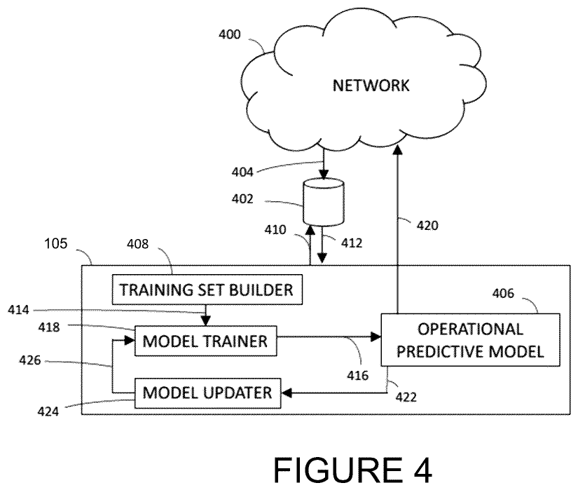

FIG. 4 is a schematic diagram of an exemplary privacy analysis (PA) server 105 (shown in FIG. 1), that may be used with the systems 100 and 300 (shown in FIGS. 1 and 3). PA server 105 may communicate with other components of system 300, such as user devices 115, requestor devices 120, internal data sources 130, external data sources 125, analysis LLMs 135 and/or privacy LLMs 140 (all shown in FIG. 1), via a network 400.

PA server 105 may also include and/or be in communication with a database 402 that stores data 404, such as database 310 (shown in FIG. 3), stored records generated by PA server 105, and/or any other relevant data s described herein. Data 404 received from network 400 may be stored in database 402. PA server 105 may configured to use data 404 to generate an operational large language model module 406 for controlling operations of PA server 105 (e.g., in accessing third-party databases via a digital portal), generating questions, timing, and language for requesting information from an individual, and the like.

In exemplary embodiments, PA server 105 may include a training set builder module 408 configured to submit one or more queries 410 to database 402 to retrieve subsets 412 of data 404, and to use those subsets 412 to build training data sets 414 for generating operational large language module 406. For example, query 410 may be configured to retrieve certain fields from data 404 for specific information, specific product, specific category, and/or any other division of factors desired by the user and/or for compliance, such as with a government entity.

In various embodiments, training set builder module 408 may be configured to derive training data sets 414 from retrieved subsets 412. Each training data set 414 corresponds to a historical data 404 (“historical” in this context means completed in the past, as opposed to completed in real-time with respect to the time of retrieval). Each training data set 414 may include “model input” data fields along with at least one “result” data field representing a historical outcome associated with the model input. The model input data fields represent factors that may be expected to, or unexpectedly be found during model training to, have some correlation.

In exemplary embodiments, the model input data fields in training data sets 414 may be generated from data fields in subset 412 corresponding to historical data 404. In other words, a trained machine learning model 416 produced by a model trainer module 418 for use by operational predictive model module 406 is trained to make predictions based upon input values that can be generated from the data fields in data 404. Values in the model input data fields may include values copied directly from values in a corresponding data field in the retrieved subset 412, and/or values generated by modifying, combining, or otherwise operating upon values in one or more data fields in the retrieved subset 412. The use of such data fields as model input data fields facilitates the machine learning model in weighing these factors directly.

After training set builder module 408 generates training data sets 414, training set builder module 408 passes the training data sets 414 to model trainer module 418. In certain embodiments, model trainer module 418 may be configured to apply the model input data fields of each training data set 414 as inputs to one or more machine learning models. Each of the one or more machine learning models may be programmed to produce, for cach training data set 414, at least one output intended to correspond to, or “predict,” a value of the at least one result data field of the training data set 414. “Machine learning” refers broadly to various algorithms that may be used to train the model to identify and recognize patterns in existing data in order to facilitate making predictions for subsequent new input data.

Model trainer module 418 may be configured to compare, for each training data set 414, the at least one output of the model to the at least one result data field of the training data set 414, and apply a machine learning algorithm to adjust parameters of the model in order to reduce the difference or “error” between the at least one output and the corresponding at least one result data field. In this way, model trainer module 418 trains the machine learning model to accurately predict the value of the at least one result data field.

In other words, model trainer module 418 cycles the one or more machine learning models through the training data sets 414, causing adjustments in the model parameters, until the error between the at least one output and the at least one result data field falls below a suitable threshold, and then uploads at least one trained machine learning model 416 to operational large language model module 406 for application to generating recommendations 420. In exemplary embodiments, model trainer module 418 may be configured to simultaneously train multiple candidate machine learning models and to select the best performing candidate for each result data field, as measured by the “error” between the at least one output and the corresponding result data field, to upload to operational predictive model module 406.

In certain embodiments, the one or more machine learning models may include one or more neural networks, such as a convolutional neural network, a deep learning neural network, or the like. The neural network may have one or more layers of nodes, and the model parameters adjusted during training may be respective weight values applied to one or more inputs to each node to produce a node output. In other words, the nodes in each layer may receive one or more inputs and apply a weight to each input to generate a node output. The node inputs to the first layer may correspond to the model input data fields, and the node outputs of the final layer may correspond to the at least one output of the model, intended to predict the at least one result data field. One or more intermediate layers of nodes may be connected between the nodes of the first layer and the nodes of the final layer.

As model trainer module 418 cycles through the training data sets 414, model trainer module 418 applies a suitable backpropagation algorithm to adjust the weights in each node layer to minimize the error between the at least one output and the corresponding result data field. In this fashion, the machine learning model is trained to produce output that reliably predicts the corresponding result data field. Alternatively, the machine learning model may have any suitable structure.

In some embodiments, model trainer module 418 may provide an advantage by automatically discovering and properly weighting complex, second-or third-order, and/or otherwise nonlinear interconnections between the model input data fields and the at least one output. Absent the machine learning model, such connections are unexpected and/or undiscoverable by human analysts.

The PA server 105 of the present disclosure may be configured to operate on input data related to privacy interactions including analyzing past privacy settings to determine effective privacy settings. In one exemplary embodiment, PA server 105 executes the operational large language model module 406 programmed to learn, without limitation, different healthcare issues that may arise and the indicators that are early signs of those issues.

To facilitate this learning, PA server 105 may include one or more databases 402 at which the data, including data as well as responses, evidence, outcomes, etc., is stored. This data becomes one or more input training sets used by the training set builder module 408. Model outputs can be formatted for presentation or review as visual representations of recommendations, as text-based or natural language recommendations, and the like.

In exemplary embodiments, operational large language model module 406 may compare feedback, and may route a comparison result 422 generated by comparing recommendation 420 to the feedback to a model updater module 424 of PA server 105. Model updater module 424 is configured to derive a correction signal 426 from comparison results 422 received for one or more recommendations, and to provide correction signal 426 to model trainer module 418 to enable updating or “re-training” of the at least one machine learning model to improve performance. The retrained at least one machine learning model 416 may be periodically re-uploaded to operational large language model module 406.

### Exemplary Client Device

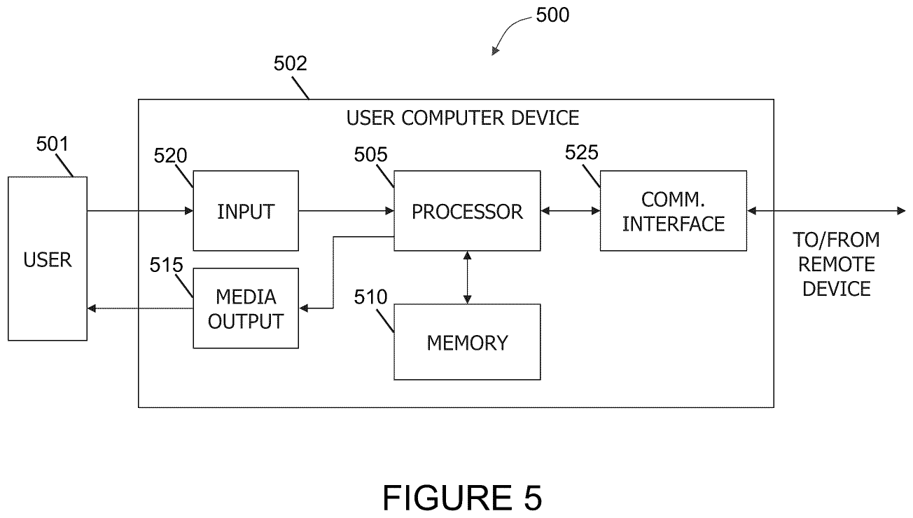

FIG. 5 depicts an exemplary configuration 500 of user computer device 502, in accordance with one embodiment of the present disclosure. In the exemplary embodiment, user computer device 502 may be similar to, or the same as, user device 115 (shown in FIG. 1) and requestor device 120 (shown in FIG. 1). User computer device 502 may be operated by a user 501.

User computer device 502 may include a processor 505 for executing instructions. In some embodiments, executable instructions may be stored in a memory area 510. Processor 505 may include one or more processing units (e.g., in a multi-core configuration). Memory area 510 may be any device allowing information such as executable instructions and/or transaction data to be stored and retrieved. Memory area 510 may include one or more computer readable media.

User computer device 502 may also include at least one media output component 515 for presenting information to user 501. Media output component 515 may be any component capable of conveying information to user 501. In some embodiments, media output component 515 may include an output adapter (not shown) such as a video adapter and/or an audio adapter. An output adapter may be operatively coupled to processor 505 and operatively couplable to an output device such as a display device (e.g., a cathode ray tube (CRT), liquid crystal display (LCD), light emitting diode (LED) display, or “electronic ink” display) or an audio output device (e.g., a speaker or headphones).

In some embodiments, media output component 515 may be configured to present a graphical user interface (e.g., a web browser and/or a client application) to user 501. A graphical user interface may include, for example, an interface for viewing items of information provided by the PA computer device 105 (shown in FIG. 1). In some embodiments, user computer device 502 may include an input device 520 for receiving input from user 501. User 501 may use input device 520 to, without limitation, provide information either through speech or typing.

Input device 520 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 515 and input device 520.

User computer device 502 may also include a communication interface 525, communicatively coupled to a remote device such as PA computer device 105. Communication interface 525 may include, for example, a wired or wireless network adapter and/or a wireless data transceiver for use with a mobile telecommunications network.

Stored in memory area 510 are, for example, computer readable instructions for providing a user interface to user 501 via media output component 515 and, optionally, receiving and processing input from input device 520. A user interface may include, among other possibilities, a web browser and/or a client application. Web browsers enable users, such as user 501, to display and interact with media and other information typically embedded on a web page or a website from PA computer device 105. A client application may allow user 501 to interact with, for example, PA computer device 105. For example, instructions may be stored by a cloud service, and the output of the execution of the instructions sent to the media output component 515.

### Exemplary Server Device

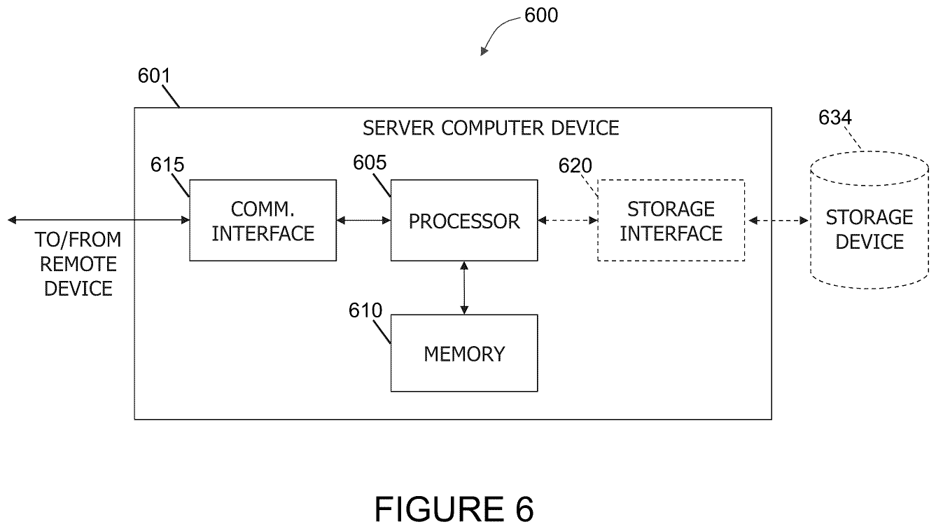

FIG. 6 depicts an exemplary configuration 600 of a server computer device 601, in accordance with one embodiment of the present disclosure. In the exemplary embodiment, server computer device 601 may be similar to, or the same as, PA computer device 105, requestor device 120 (both shown in FIG. 1), and database server 305 (shown in FIG. 3). Server computer device 601 may also include a processor 605 for executing instructions. Instructions may be stored in a memory area 610. Processor 605 may include one or more processing units (e.g., in a multi-core configuration).

Processor 605 may be operatively coupled to a communication interface 615 such that server computer device 601 is capable of communicating with a remote device such as another server computer device 601, PA computer device 105, requestor devices 120, and user devices 115 (shown in FIG. 1) (for example, using wireless communication or data transmission over one or more radio links or digital communication channels). For example, communication interface 615 may audio input from user devices 115 via the Internet, as illustrated in FIG. 3.

Processor 605 may also be operatively coupled to a storage device 634. Storage device 634 may be any computer-operated hardware suitable for storing and/or retrieving data, such as, but not limited to, data associated with one or more models. In some embodiments, storage device 634 may be integrated in server computer device 601. For example, server computer device 601 may include one or more hard disk drives as storage device 634.

In other embodiments, storage device 634 may be external to server computer device 601 and may be accessed by a plurality of server computer devices 601. For example, storage device 634 may include a storage area network (SAN), a network attached storage (NAS) system, and/or multiple storage units such as hard disks and/or solid-state disks in a redundant array of inexpensive disks (RAID) configuration.

In some embodiments, processor 605 may be operatively coupled to storage device 634 via a storage interface 620. Storage interface 620 may be any component capable of providing processor 605 with access to storage device 634. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 634.

Processor 605 may execute computer-executable instructions for implementing aspects of the disclosure. In some embodiments, the processor 605 may be transformed into a special purpose microprocessor by executing computer-executable instructions or by otherwise being programmed. For example, the processor 605 may be programmed with the instruction such as illustrated in FIG. 2.

### Machine Learning and Other Matters

The computer-implemented methods discussed herein may include additional, less, or alternate actions, including those discussed elsewhere herein. The methods may be implemented via one or more local or remote processors, transceivers, servers, and/or sensors (such as processors, transceivers, servers, and/or sensors mounted on vehicles or mobile devices, or associated with smart infrastructure or remote servers), and/or via computer-executable instructions stored on non-transitory computer-readable media or medium.

In some embodiments, PA computer system 105 is configured to implement machine learning, such that PA computer system 105 “learns” to analyze, organize, and/or process data without being explicitly programmed. Machine learning may be implemented through machine learning methods and algorithms (“ML methods and algorithms”). In an exemplary embodiment, a machine learning module (“ML module”) is configured to implement ML methods and algorithms.

In some embodiments, ML methods and algorithms are applied to data inputs and generate machine learning outputs (“ML outputs”). Data inputs may include but are not limited to images. ML outputs may include, but are not limited to identified objects, items classifications, and/or other data extracted from the images. In some embodiments, data inputs may include certain ML outputs.

In certain embodiments, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of machine learning, such as supervised learning, unsupervised learning, and reinforcement learning.

In one embodiment, the ML module employs supervised learning, which involves identifying patterns in existing data to make predictions about subsequently received data. Specifically, the ML module is “trained” using training data, which includes example inputs and associated example outputs. Based upon the training data, the ML module may generate a predictive function which maps outputs to inputs and may utilize the predictive function to generate ML outputs based upon data inputs. The example inputs and example outputs of the training data may include any of the data inputs or ML outputs described above. In the exemplary embodiment, a processing element may be trained by providing it with a large sample of images with known characteristics or features. Such information may include, for example, information associated with a plurality of images of a plurality of different objects, items, property and/or health-related characteristics of individuals.

In another embodiment, a ML module may employ unsupervised learning, which involves finding meaningful relationships in unorganized data. Unlike supervised learning, unsupervised learning does not involve user-initiated training based upon example inputs with associated outputs. Rather, in unsupervised learning, the ML module may organize unlabeled data according to a relationship determined by at least one ML method/algorithm employed by the ML module. Unorganized data may include any combination of data inputs and/or ML outputs as described above.

In yet another embodiment, a ML module may employ reinforcement learning, which involves optimizing outputs based upon feedback from a reward signal. Specifically, the ML module may receive a user-defined reward signal definition, receive a data input, utilize a decision-making model to generate a ML output based upon the data input, receive a reward signal based upon the reward signal definition and the ML output, and alter the decision-making model so as to receive a stronger reward signal for subsequently generated ML outputs. Other types of machine learning may also be employed, including deep or combined learning techniques.

In some embodiments, generative artificial intelligence (AI) models (also referred to as generative machine learning (ML) models) may be utilized with the present embodiments and may the voice bots or chatbots discussed herein may be configured to utilize artificial intelligence and/or machine learning techniques. For instance, the voice or chatbot may be a ChatGPT chatbot. The voice or chatbot may employ supervised or unsupervised machine learning techniques, which may be followed by, and/or used in conjunction with, reinforced or reinforcement learning techniques. The voice or chatbot may employ the techniques utilized for ChatGPT. The voice bot, chatbot, ChatGPT-based bot, ChatGPT bot, and/or other bots may generate audible or verbal output, text or textual output, visual or graphical output, output for use with speakers and/or display screens, and/or other types of output for user and/or other computer or bot consumption.

Based upon these analyses, the processing element may learn how to identify characteristics and patterns that may then be applied to analyzing and classifying objects, and/or individuals for providing secure information and/or health-related characteristics. The processing element may also learn how to identify attributes of different objects in different lighting. This information may be used to determine which classification models to use and which classifications to provide.

### Exemplary Embodiments

In one aspect, a computer system may be provided. The computer system may include one or more local or remote processors, servers, sensors, memory units, transceivers, mobile devices, wearables, smart watches, smart glasses or contacts, augmented reality glasses, virtual reality headsets, mixed or extended reality headsets, voice bots, chatbots, ChatGPT bots, and/or other electronic or electrical components, which may be in wired or wireless communication with one another. For instance, the computer system may include at least one processor in communication with at least one memory device. The at least one processor may be configured to: (1) store a plurality of sets of personal information for a plurality of individuals, wherein each set of personal information of the plurality of personal information is stored with a plurality of privacy settings for accessing the corresponding set of personal information; (2) receive, from a requestor device, a request for access to a first set of personal information for a first individual, wherein the request for access includes one or more attributes; (3) compare the one or more attributes of the request for access to the plurality of privacy settings for the first set of personal information; (4) determine one or more items of information from the first set of personal information approved to be provided in response to the request for access; (5) generate a response to the request for access to the first set of personal information including the one or more items of information; and/or (6) transmit, to the requestor device, the response to the request for access to the first set of personal information. The system may include additional, less, or alternate functionality, including that discussed elsewhere herein.

An enhancement of the system may include a processor configured to analyze the plurality of personal information. The personal information may be, for instance, retrieved from one or more memory units and/or acquired via one or more sensors, including microphones, mobile devices, AR or VR headsets or glasses, smart glasses, wearables, smart watches, or other electronic or electrical devices; and/or acquired via, or at the direction of, generative AI or machine learning models, such as at the direction of bots, such as ChatGPT bots, or other chat or voice bots, interconnected with one or more sensors, including cameras or video recorders.

An enhancement of the system may include a processor configured to analyze the plurality of privacy interactions. The privacy interactions may be, for instance, retrieved from one or more memory units and/or acquired via one or more sensors, including microphones, mobile devices, AR or VR headsets or glasses, smart glasses, wearables, smart watches, or other electronic or electrical devices; and/or acquired via, or at the direction of, generative AI or machine learning models, such as at the direction of bots, such as ChatGPT bots, or other chat or voice bots, interconnected with one or more sensors, including cameras or video recorders.

A further enhancement of the system may include a processor configured to execute a privacy model to determine the one or more items of information from the first set of personal information to approve providing in response to the request for access. The system may further train the privacy model to determine an amount of access to provide to requestors based upon a plurality of historical requests and responses. The system may also determine one or more of the plurality of privacy settings based upon execution of the privacy model.

A further enhancement of the system may include where the plurality of personal information includes personal healthcare information (PHI). A further enhancement of the system may include a processor configured to analyze a set of personal information to determine a healthcare recommendation for the corresponding individual. The system may further provide the healthcare recommendation to the corresponding individual.

A further enhancement of the system may include where the requestor device executes an application programming interface (API) to transmit the request for access.

A further enhancement of the system may include where the one or more attributes includes a category for a requestor associated with the requestor device. A further enhancement of the system may include a processor configured to determine one or more items of information from the first set of personal information to provide in response to the request for access based upon the category for the requestor.

A further enhancement of the system may include where the one or more attributes includes a category for a requestor associated with the requestor device. A further enhancement of the system may include a processor configured to determine one or more items of information from the first set of personal information to prevent access to based upon the category for the requestor.

A further enhancement of the system may include a processor configured to receive the plurality of privacy settings for a set of personal information from the corresponding individual.

A further enhancement of the system may include a processor configured to analyze a set of personal information to determine a service to provide to the corresponding individual.

In another aspect, a computer-implemented method may be provided. The computer-implemented method may be performed by a computer device including at least one processor in communication with at least one memory device. The method may include: (1) storing a plurality of sets of personal information for a plurality of individuals, wherein each set of personal information of the plurality of personal information is stored with a plurality of privacy settings for accessing the corresponding set of personal information; (2) receiving, from a requestor device, a request for access to a first set of personal information for a first individual, wherein the request for access includes one or more attributes; (3) comparing the one or more attributes of the request for access to the plurality of privacy settings for the first set of personal information; (4) determining one or more items of information from the first set of personal information approved to be provided in response to the request for access; (5) generating a response to the request for access to the first set of personal information including the one or more items of information; and/or (6) transmitting, to the requestor device, the response to the request for access to the first set of personal information. The computer-implemented method may include additional, less, or alternate actions, including those discussed elsewhere herein.

An enhancement of the method may include analyzing a plurality of personal information. The personal information may be, for instance, retrieved from one or more memory units and/or acquired via one or more sensors, including cameras, microphones, mobile devices, AR or VR headsets or glasses, smart glasses, wearables, smart watches, or other electronic or electrical devices; and/or acquired via, or at the direction of, generative AI or machine learning models, such as at the direction of bots, such as ChatGPT bots, or other chat or voice bots, interconnected with one or more sensors, including cameras or video recorders.

An enhancement of the method may include analyzing a plurality of privacy interactions. The privacy interactions may be, for instance, retrieved from one or more memory units and/or acquired via one or more sensors, including cameras, microphones, mobile devices, AR or VR headsets or glasses, smart glasses, wearables, smart watches, or other electronic or electrical devices; and/or acquired via, or at the direction of, generative AI or machine learning models, such as at the direction of bots, such as ChatGPT bots, or other chat or voice bots, interconnected with one or more sensors, including cameras or video recorders.

An enhancement of the computer-implemented method may include executing a privacy model to determine the one or more items of information from the first set of personal information to approve providing in response to the request for access. Additionally or alternatively, a further enhancement of the computer-implemented method may include determine an amount of access to provide to requestors based upon a plurality of historical requests and responses. Additionally or alternatively, an additional enhancement of the computer-implemented method may include determining one or more of the plurality of privacy settings based upon execution of the privacy model.

An enhancement of the computer-implemented method may include where the plurality of personal information includes personal healthcare information (PHI). An enhancement of the computer-implemented method may also include analyzing a set of personal information to determine a healthcare recommendation for the corresponding individual. An enhancement of the computer-implemented method may further include providing the healthcare recommendation to the corresponding individual.

An enhancement of the computer-implemented method may include where the requestor device executes an application programming interface (API) to transmit the request for access.

An enhancement of the computer-implemented method may include where the one or more attributes includes a category for a requestor associated with the requestor device. An enhancement of the computer-implemented method may include determining one or more items of information from the first set of personal information to provide in response to the request for access based upon the category for the requestor.

An enhancement of the computer-implemented method may include where the one or more attributes includes a category for a requestor associated with the requestor device. An enhancement of the computer-implemented method may include determining one or more items of information from the first set of personal information to prevent access to based upon the category for the requestor.

An enhancement of the computer-implemented method may include receiving the plurality of privacy settings for a set of personal information from the corresponding individual.

An enhancement of the computer-implemented method may include analyzing a set of personal information to determine a service to provide to the corresponding individual.

In another aspect, at least one non-transitory computer-readable media having computer-executable instructions embodied thereon may be provided. When executed by a computing device including at least one processor in communication with at least one memory device, the computer-executable instructions may cause the at least one processor to: (1) store a plurality of sets of personal information for a plurality of individuals, wherein each set of personal information of the plurality of personal information is stored with a plurality of privacy settings for accessing the corresponding set of personal information; (2) receive, from a requestor device, a request for access to a first set of personal information for a first individual, wherein the request for access includes one or more attributes; (3) compare the one or more attributes of the request for access to the plurality of privacy settings for the first set of personal information; (4) determine one or more items of information from the first set of personal information approved to be provided in response to the request for access; (5) generate a response to the request for access to the first set of personal information including the one or more items of information; and/or (6) transmit, to the requestor device, the response to the request for access to the first set of personal information. The computer-executable instructions may direct additional, less, or alternate functionality, including that discussed elsewhere herein.

### Additional Considerations

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps,” or code) include machine instructions for a programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, the term “database” can refer to either a body of data, a relational database management system (RDBMS), or to both. As used herein, a database can include any collection of data including hierarchical databases, relational databases, flat file databases, object-relational databases, object-oriented databases, and any other structured collection of records or data that is stored in a computer system. The above examples are example only, and thus are not intended to limit in any way the definition and/or meaning of the term database. Examples of RDBMS' include, but are not limited to including, Oracle® Database, MySQL, NoSQL, IBMR DB2, Microsoft® SQL Server, and PostgreSQL. However, any database can be used that enables the systems and methods described herein. (Oracle is a registered trademark of Oracle Corporation, Redwood Shores, California; IBM is a registered trademark of International Business Machines Corporation, Armonk, New York; and Microsoft is a registered trademark of Microsoft Corporation, Redmond, Washington.)

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are example only and are thus not limiting as to the types of memory usable for storage of a computer program.

In another example, a computer program is provided, and the program is embodied on a computer-readable medium. In an example, the system is executed on a single computer system, without requiring a connection to a server computer. In a further example, the system is being run in a Windows® environment (Windows is a registered trademark of Microsoft Corporation, Redmond, Washington). In yet another example, the system is run on a mainframe environment and a UNIX® server environment (UNIX is a registered trademark of X/Open Company Limited located in Reading, Berkshire, United Kingdom). In a further example, the system is run on an iOS® environment (iOS is a registered trademark of Cisco Systems, Inc. located in San Jose, CA). In yet a further example, the system is run on a Mac OS® environment (Mac OS is a registered trademark of Apple Inc. located in Cupertino, CA). In still yet a further example, the system is run on Android® OS (Android is a registered trademark of Google, Inc. of Mountain View, CA). In another example, the system is run on Linux® OS (Linux is a registered trademark of Linus Torvalds of Boston, MA). The application is flexible and designed to run in various different environments without compromising any major functionality.

In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, an element or step recited in the singular and proceeded with the word “a” or “an” should be understood as not excluding plural elements or steps, unless such exclusion is explicitly recited. Furthermore, references to “example” or “one example” of the present disclosure are not intended to be interpreted as excluding the existence of additional examples that also incorporate the recited features. Further, to the extent that terms “includes,” “including,” “has,” “contains,” and variants thereof are used herein, such terms are intended to be inclusive in a manner similar to the term “comprises” as an open transition word without precluding any additional or other elements.

Furthermore, as used herein, the term “real-time” refers to at least one of the time of occurrence of the associated events, the time of measurement and collection of predetermined data, the time to process the data, and the time of a system response to the events and the environment. In the examples described herein, these activities and events occur substantially instantaneously.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer system for advanced provisioning of secure information, the system comprising at least one processor in communication with at least one memory device, the at least one processor programmed to:
store a plurality of sets of personal information for a plurality of individuals, wherein each set of personal information of the plurality of personal information is stored with a plurality of privacy settings for accessing the corresponding set of personal information;
receive, from a requestor device, a request for access to a first set of personal information for a first individual, wherein the request for access includes one or more attributes;
compare the one or more attributes of the request for access to the plurality of privacy settings for the first set of personal information;
determine one or more items of information from the first set of personal information approved to be provided in response to the request for access;
generate a response to the request for access to the first set of personal information including the one or more items of information; and
transmit, to the requestor device, the response to the request for access to the first set of personal information.

2. The computer system of claim 1, wherein the at least one processor is further programmed to execute a privacy model to determine the one or more items of information from the first set of personal information to approve providing in response to the request for access.

3. The computer system of claim 2, wherein the at least one processor is further programmed to train the privacy model to determine an amount of access to provide to requestors based upon a plurality of historical requests and responses.

4. The computer system of claim 2, wherein the at least one processor is further programmed to determine one or more of the plurality of privacy settings based upon execution of the privacy model.

5. The computer system of claim 1, wherein the plurality of personal information includes personal healthcare information (PHI).

6. The computer system of claim 5, wherein the at least one processor is further programmed to:
analyze a set of personal information to determine a healthcare recommendation for the corresponding individual; and
provide the healthcare recommendation to the corresponding individual.

7. The computer system of claim 1, wherein the requestor device executes an application programming interface (API) to transmit the request for access.

8. The computer system of claim 1, wherein the one or more attributes includes a category for a requestor associated with the requestor device, and wherein the at least one processor is further programmed to determine one or more items of information from the first set of personal information to be provided in response to the request for access based upon the category for the requestor.

9. The computer system of claim 1, wherein the one or more attributes includes a category for a requestor associated with the requestor device, and wherein the at least one processor is further programmed to determine one or more items of information from the first set of personal information to prevent access to based upon the category for the requestor.

10. The computer system of claim 1, wherein the at least one processor is further programmed to receive the plurality of privacy settings for a set of personal information from the corresponding individual.

11. The computer system of claim 1, wherein the at least one processor is further programmed to analyze a set of personal information to determine a service to provide to the corresponding individual.

12. A computer-implemented method for advanced provisioning of secure information that is implemented by a computer system including at least one processor in communication with at least one memory device, the method comprises:
storing a plurality of sets of personal information for a plurality of individuals, wherein each set of personal information of the plurality of personal information is stored with a plurality of privacy settings for accessing the corresponding set of personal information;
receiving, from a requestor device, a request for access to a first set of personal information for a first individual, wherein the request for access includes one or more attributes;
comparing the one or more attributes of the request for access to the plurality of privacy settings for the first set of personal information;
determining one or more items of information from the first set of personal information approved to be provided in response to the request for access;
generating a response to the request for access to the first set of personal information including the one or more items of information; and
transmitting, to the requestor device, the response to the request for access to the first set of personal information.

13. The computer-implemented method of claim 12 further comprising executing a privacy model to determine the one or more items of information from the first set of personal information to approve providing in response to the request for access.

14. The computer-implemented method of claim 13 further comprising training the privacy model to determine an amount of access to provide to requestors based upon a plurality of historical requests and responses.

15. The computer-implemented method of claim 13 further comprising determining one or more of the plurality of privacy settings based upon execution of the privacy model.

16. The computer-implemented method of claim 12, wherein the plurality of personal information includes personal healthcare information (PHI).

17. The computer-implemented method of claim 16 further comprising:
analyzing a set of personal information to determine a healthcare recommendation for the corresponding individual; and
providing the healthcare recommendation to the corresponding individual.

18. The computer-implemented method of claim 12, wherein the one or more attributes includes a category for a requestor associated with the requestor device, and wherein the method further comprises determining one or more items of information from the first set of personal information to provide in response to the request for access based upon the category for the requestor.

19. The computer-implemented method of claim 12, wherein the one or more attributes includes a category for a requestor associated with the requestor device, and wherein the method further comprises determining one or more items of information from the first set of personal information to prevent access to based upon the category for the requestor.

20. The computer-implemented method of claim 12 further comprising analyzing a set of personal information to determine a service to provide to the corresponding individual.

21. At least one non-transitory computer-readable storage media having computer-executable instructions embodied thereon, wherein when executed by at least one processor of a computer system, the computer-executable instructions cause the processor to:
store a plurality of sets of personal information for a plurality of individuals, wherein each set of personal information of the plurality of personal information is stored with a plurality of privacy settings for accessing the corresponding set of personal information;
receive, from a requestor device, a request for access to a first set of personal information for a first individual, wherein the request for access includes one or more attributes;
compare the one or more attributes of the request for access to the plurality of privacy settings for the first set of personal information;
determine one or more items of information from the first set of personal information approved to be provided in response to the request for access;
generate a response to the request for access to the first set of personal information including the one or more items of information; and
transmit, to the requestor device, the response to the request for access to the first set of personal information.
